(19) United States
(12) Patent Application Publication
(10) Pub. No.: US 2023/0174462 A1
(43) Pub. Date: Jun. 8, 2023

(54) METHOD FOR THE CONTINUOUS SYNTHESIS OF PARACETAMOL

(71) Applicant: IPSOMEDIC, MARSEILLE (FR)

(72) Inventors: Edith LECOMTE-NORRANT, LA CIOTAT (FR); Romain MEMBRAT, MARSEILLE (FR)

(73) Assignee: IPSOMEDIC, MARSEILLE (FR)

(21) Appl. No.: 17/921,177

(22) PCT Filed: Apr. 27, 2021
(86) PCT No.: PCT/EP2021/061000

§ 371 Date: Oct. 25, 2022

(30) Foreign Application Priority Data
Apr. 27, 2020 (FR) 2004184
Nov. 23, 2020 (FR) 2012032

(51) Int. Cl. C07C 231/22

(52) U.S. Cl. CPC C07C 231/22

(57) ABSTRACT

A continuous paracetamol preparation method, including a nitration step or a nitrosation step to obtain p-nitrophenol or p-nitrosophenol respectively. P-nitrophenol or p-nitrosophenol can then be converted into paracetamol by hydrogenation, followed by acylation. This continuous paracetamol preparation method makes it possible to obtain paracetamol with a very good regioselectivity and excellent yields.

### TECHNICAL FIELD

The present invention relates to a method for the continuous synthesis of paracetamol.

### PRIOR ART

Paracetamol, also called acetaminophen, corresponds to N(4-hydroxyphenyl)0 acetamide. This compound is used as both analgesic (painkiller) and antipyretic (anti-fever), and is among the most common, used and prescribed drugs in the world.

It is indicated in the treatment of low to moderate pain and its great popularity is due to the fact that it has fewer contraindications than other analgesics and enjoys a good image among the public.

The exact mechanism by which acetaminophen produces its analgesic and antipyretic effects remains undefined. The main mechanism of action is thought to be the inhibition of cyclooxygenase (COX), with a predominant effect on COX-2.

There are multiple paracetamol synthesis methods, such as those described in patents EP 0,435,263, US 6,969,775, and EP 2,266,949.

FIG. 1 summarizes the main chemical pathways of paracetamol synthesis with their intermediates.

For the most part, these pathways pass through phenol (via cumene), chlorobenzene or nitrobenzene, all of which are high-tonnage raw materials worldwide.

Starting from nitrobenzene, 3 chemical steps are necessary, which correspond to hydrogenation, followed by hydroxylation and acylation to obtain paracetamol.

Starting from chlorobenzene, it takes 4 chemical steps. In this case, the synthesis begins with a nitration, followed by hydrolysis, reduction and at last acylation to obtain paracetamol.

At last, with regard to phenol, two pathways are possible, which involve two or three steps respectively. The two-stage pathway passes through hydroquinone, but is relatively complex (reaction of more than 12 hours at 200° C. with purification between the two stages due to solvent change). As for the 3-step pathway, it goes through 4-aminophenol.

This last pathway, which is among the most used, goes through a nitration of phenol with the formation of para-nitrophenol and is followed by a reduction and acetylation of the latter to form paracetamol.

Now, this synthetic pathway suffers from a fundamental shortcoming which lies in the fact that the step of nitration of phenol to obtain the p-nitrophenol of interest - which is the precursor of paracetamol - has a low yield. Indeed, the product of the reaction in the ortho position of the phenyl ring may have a proportion of up to 66%, this constituting the normal result of the fact that one has two equivalent attachment positions resulting in o-nitrophenol for only one position for the para position. In addition, o-nitrophenol is favored by the formation of a hydrogen bond between the hydroxyl group and one of the two oxygen atoms of the NO₂ group.

As a result, different technologies have been developed to try to increase the yield of p-nitrophenol (two-step reaction giving a yield of 48% described in patent EP 0 626 366).

Now, whether it is this synthetic route or the others, it takes a production time of the order of one to two weeks to obtain purified paracetamol, and this with a significant amount of waste.

### DESCRIPTION OF THE INVENTION

The inventors have now developed a new method for the synthesis of paracetamol that is carried out continuously and which makes it possible to carry out its production in less than 3 hours, with simultaneously a considerable reduction in the amount of associated waste due to its very high efficiency.

In the batch production method, the transition from one step to the next one is carried out in series and therefore the overall method time is, in fact, the sum of the times required for the different steps.

In its general principle, this method consists of a continuous flow of integrated reactions in which a succession of reactors are interconnected. Each reactor allows the realization of a specific and essential step to arrive at the final product.

In the continuous method, all steps are performed simultaneously (although in different compartments of the system), and therefore the overall time required for the method is shortened. In addition, the volume required for reactors is much less for a continuous method which, in addition to facilitating the management of plant safety, makes it possible to work in much more restrictive (astringent) conditions than for batch methods.

A first object of the present invention relates to a method for preparing paracetamol, wherein the method comprises a step A of nitration, or nitrosation, of a compound of Formula 1 with a nitration agent, or a nitrosating agent suitable for obtaining a compound of Formula 2:

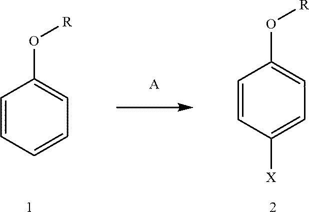

wherein R represents:
   a hydrogen atom,
   a protective group selected from a benzyl or acetate,
   and wherein X represents a nitro group or a nitroso group,
said step A of nitration being carried out continuously:
   either under microwaves,
   either under ultrasound,
   either under microwave followed by ultrasound,
   or, optionally under microwaves and / or ultrasound, the nitration agent being sodium nitrite, in the presence of an oxidizing agent, in particular in the presence of HNO₃,
said step A of nitrosation being carried out continuously:
   the nitrosating agent being sodium nitrite.

The inventors found that the continuous nitration or nitrosation reaction of phenol, optionally protected on the hydroxyl function by an acetate or benzyl group, leads respectively to the compound para-nitro, or para-nitroso, with an excellent regioselectivity. The continuous flow reaction thus makes it possible to limit the formation of the ortho isomer, as well as that of other impurities such as polymerization products.

The inventors also found that, in the context of nitration, the continuous method can be carried out in combination with microwave and/or ultrasonic irradiation, allowing improved reaction efficiency in terms of reaction rate and reduction of the formation of impurities such as the ortho isomer.

In addition, the use of a phenolic compound protected on the hydroxyl function makes it possible to introduce a steric hindrance, which, in combination with the suppression of the hydroxyl function which plays a role in the nitration mechanism, further the suppression of the formation of the ortho isomer.

According to a particular embodiment, the present invention relates to a method as defined above, wherein step A is a nitration step, R being as defined above, and X being a nitro group, to obtain a nitro compound wherein the compound of Formula 2 has the structure of a compound of Formula 2a:

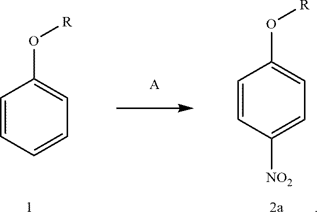

According to another particular embodiment, the present invention relates to a method as defined above, wherein step A is a nitrosation step, R being as defined above 1, and X being a nitroso group, to obtain a nitroso compound wherein the compound of Formula 2 has the structure of a compound of Formula 2b:

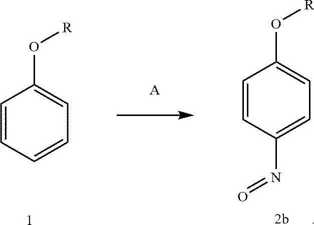

In this embodiment, a nitroso compound is obtained, which represents an intermediate that can be used in the preparation of paracetamol. In this context, it is possible to reduce the nitroso function directly in amine function, or to pre-convert the nitroso function into nitro, during an additional oxidation step, in particular by nitric acid.

The inventors observed that the nitrosation reaction is very efficient in terms of kinetics, and typically leads to a total conversion only after 10 minutes of reaction, and this with an excellent regioselectivity of 90% in favor of the para compound compared to the ortho secondary product. This nitrosation pathway is particularly advantageous because the concomitant formation of polymers is very limited.

According to another particular embodiment, the present invention relates to a method as defined above, wherein step A of nitration, or step A of nitrosation is carried out under inert atmosphere, or in the open air.

By “inert atmosphere” is meant that the nitration or nitrosation is carried out in a reactor under nitrogen or argon. On the other hand, “in the open air” means that no precautions have been taken in this direction.

According to another particular embodiment, the present invention relates to a method as defined above, wherein the compound of Formula 1 is phenol, R being a hydrogen atom, to obtain p-nitrophenol as a compound of Formula 2, X being a nitro group, or p-nitrosophenol as a compound of Formula 2, where X is a nitroso group.

In this embodiment, phenol is not protected on hydroxyl function. This represents an advantage in terms of production cost, phenol being a commodity product.

According to another particular embodiment, the present invention relates to a method as defined above, wherein step A of nitration is carried out with a nitration agent selected from HNO₃ and NaNO₂, to obtain a compound of Formula 2 wherein X is a nitro group.

Nitric acid can be used in the presence of an acid such as sulfuric acid, but also in the absence of said acid. Preferably, nitric acid is introduced into the reactor in the form of an aqueous solution.

Sodium nitrite leads to the nitro compound when used in combination with an oxidizing agent, including nitric acid.

According to another particular embodiment, the present invention relates to a method as defined above, wherein step A of nitration comprises:
a) a feeding system of a reactor with a solution, in particular aqueous, of the compound of Formula 1, in particular in concentration of about 0.4 M, and with the nitration agent, in solution, in particular aqueous, in particular at a concentration of about 0.3 to 0.4 M, to obtain a reaction medium,
b) the formation of the Formula 2 compound.

The raw materials, namely the compound of Formula 1, in solution, and reactive, namely the nitration agent, in solution, are continuously introduced into a reactor, through inlet pathways. The nitration reaction then takes place within the reactor, resulting in the formation of the compound of Formula 2, in which X represents a nitro group.

The reaction mixture, comprising the nitro product (raw product) can then be evacuated from the reactor by an output pathway.

The reactor is perfectly agitated, allowing a homogeneous distribution of materials within the reactor. Preferably, the rate of introduction or injection of the reactants is identical to the rate of evacuation or extrusion of the reaction crude, thus allowing a constant volume within the reactor.

During step A of nitration, the reactants are introduced into the reactor at a rate of 5 to 20 ml / minute, in particular about 10 ml / minute or about 15 ml / minute.

According to another particular embodiment, the present invention relates to a method as defined above, wherein step A of nitration is carried out with an initial ratio of nitration agent / compound of Formula 1 comprised from 1.1 to 1.6, preferably from 1.2 to 1.5.

By “From 1.1 to 1.6” is also meant the following ranges: from 1.1 to 1.5, from 1.1 to 1.4, from 1.1 to 1.3, from 1.1 to 1.2, from 1.2 to 1.6, from 1.3 to 1.6, from 1.4 to 1.6, from 1.5 to 1.6, from 1.2 to 1.5, from 1.3 to 1.4.

The “initial ratio” refers to the ratio with which the nitration agent and the compound of Formula 1 are introduced into the reactor. At this stage, the reagent and the raw material have not yet been involved in the nitration reaction.

According to another particular embodiment, the present invention relates to a method as defined above, wherein step A of nitration, or nitrosation is carried out with an initial concentration of the compound of Formula 1, in particular phenol, ranging from 0.2 to 0.6M, in particular from 0.25 to 0.5 M.

According to another particular embodiment, the present invention relates to a method as defined above, wherein step A of nitration, or nitrosation is carried out with an initial concentration of nitration agent, in particular HNO₃, or NaNO₂ respectively, ranging from 0.25 to 0.8M, in particular between 0.3 to 0.7M.

According to another particular embodiment, the present invention relates to a method as defined above, wherein step A of nitration is carried out at a temperature of 70 to 110° C., preferably from 80 to 100° C.

Below 70° C., the kinetics of the reaction may be too low to be compatible with an industrially viable method. Above 110° C., secondary products, such as polymers or polynitro products, can be formed. In the case where phenol is used as a raw material, the hydroxyl function can also be nitrated, to form o-nitrophenol as a by-product.

By “70 to 110° C.” is also meant the following ranges: from 70 to 100° C., from 70 to 90° C., from 70 to 80° C., from 80 to 110° C., from 90 to 110° C., from 100 to 110° C., from 80 to 100° C.

According to another particular embodiment, the present invention relates to a method as defined above, wherein step A of nitration is carried out under microwaves, nitration under microwave being carried out in a continuous microwave with a wave generator of 2.45 GHz, or 915 MHz.

Commercially available microwaves are equipped with a 2.45 GHz or 915 MHz wave generator. In an industrial method, a frequency of 915 MHz is desirable.

According to another particular embodiment, the present invention relates to a method as defined above, which step A of nitration is carried out under microwaves, nitration under microwave being carried out in a continuous microwave with a power ranging from 200 to 1000 W.

Commercially available microwaves have a power ranging from 200 to 1000 W.

By “From 200 to 1000 W” is also meant the following ranges: from 200 to 800 W, from 200 to 600 W, from 200 to 400 W, from 400 to 1000 W, from 600 to 1000 W, from 800 to 1000 W, from 400 to 800 W. The power is in particular about 450 W, or about 850 W.

According to another particular embodiment, the present invention relates to a method as defined above, wherein step A of nitration, is carried out in the presence of a cooling means to control the temperature.

Nitration reactions are exothermic. Equipping the reactor with a cooling means makes it possible to control this exothermy. The reactor may, for example, be equipped with a double jacket allowing the circulation of a coolant.

Therefore, the expression “temperature control” means to ensure that the temperature of the reaction medium remains low enough, below 110° C., preferably below 90° C., to avoid the formation of by-products, such as the ortho isomer, and polymers.

According to another particular embodiment, the present invention relates to a method as defined above, wherein step A of nitration is carried out in a reactor equipped with a wave generator with integration of a cooling system.

In this embodiment, a microwave reactor equipped with a cooling means is used. Typically, a tube is integrated in the microwave reactor, in order to circulate a heat transfer fluid, maintained at the desired temperature thanks to a cryostat.

For example, a cooled tubular continuous reactor: cavity “DOWNSTREAM” of the company SAIREM can be used.

According to another particular embodiment, the present invention relates to a method as defined above, wherein step A of nitration is carried out in at least two microwave reactors in series, preferably at least three microwave reactors in series and particularly preferably at least four microwave reactors in series.

In this embodiment, several reactors are configured in series. The raw materials, namely the compound of Formula 1, in solution, and reactive, namely the nitration agent, in solution, are introduced into the first reactor, in which the nitration reaction takes place, with a non-total conversion. The reaction medium continuously exits the first reactor, and is injected into the subsequent reactor, in which the nitration reaction continues to take place.

For example, for a system comprising 3 reactors in series, the conversion at the end of the first reactor can be 60%, the conversion at the end of the second reactor can be 90%, and a total conversion can be observed in the third reactor.

According to another particular embodiment, the present invention relates to a method as defined above, comprising, between each reactor in series, a cooling step so as to adjust the temperature to a temperature of 20 to 40° C., preferably from 20 to 30° C.

In this embodiment, the reaction medium is cooled between 2 reactors in series. This operation makes it possible to inject a cooled mixture into the subsequent reactor, which leads to a better control of the exothermic phenomena of the reaction.

FIG. 3 schematizes this configuration with several microwave reactors in series, where each couple of microwave reactors is interconnected by a cooling circuit.

According to another particular embodiment, the present invention relates to a method as defined above, wherein step A of nitrosation is carried out in an acidic medium, in particular in an aqueous solution of hydrochloric acid or sulfuric acid.

In this embodiment, an aqueous solution of the nitrosating agent, i.e. sodium nitrite is previously acidified, in particular at a pH less than 4, by adding an acid. The mixture thus obtained is injected into the reactor.

According to another particular embodiment, the present invention relates to a method as defined above, wherein step A of nitrosation comprises:
a) a feeding system of a reactor with a solution of the compound of Formula 1, in particular aqueous, and with NaNO₂ in solution, in particular aqueous in an acid, in particular in hydrochloric acid, to obtain a reaction medium,
b) the formation of the compound of Formula 2.

During step A of nitrosation, the reagent, namely NaNO₂, in solution, and the raw material, namely the compound of Formula 1, in solution, are introduced into the reactor at a rate of 5 to 20 ml / minute, in particular about 10 ml / minute or about 15 ml / minute.

According to another particular embodiment, the present invention relates to a method as defined above, comprising a step A of nitrosation, wherein a reactor is powered by an aqueous solution of the compound of Formula 1, and by NaNO₂ in aqueous solution in an acid, in particular in hydrochloric acid.

According to another particular embodiment, the present invention relates to a method as defined above, wherein step A of nitrosation is carried out at a temperature below 10° C., in particular comprised from -5 to 5° C., in particular at a temperature of about 0° C.

Above 10° C., the reaction may have a high kinetics, which can lead to a runaway exothermic reaction. In addition, under these conditions, secondary products such as poly-nitrosated products can be formed.

According to another particular embodiment, the present invention relates to a method as defined above, wherein step A of nitrosation, is carried out in the presence of a cooling means to control the temperature.

Nitrosation reactions are exothermic. Equipping the reactor with a cooling means makes it possible to control this exothermy. The reactor may, for example, be equipped with a double jacket allowing the circulation of a coolant.

According to another particular embodiment, the present invention relates to a method as defined above, wherein step A of nitration, or nitrosation leads to the formation of the compound of Formula 2, in particular p-nitrophenol or p-nitrosophenol, in particular p-nitrosophenol, with a regioselectivity greater than 60%, in particular greater than 80%, in particular wherein the ratio of ortho/compound isomer of Formula 2 is less than 2/8, and is in particular about 1/9.

By “Regioselectivity greater than 60%” is also meant a regioselectivity greater than 70%, greater than 80%, and greater than 90%. Such regioselectivity canfor example be determined by NMR, or by HPLC.

At the end of step A of nitration, or nitrosation, the reaction crude product can be directly carried into the reactor of the next step, i.e. the hydrogenation step. However, it is more advantageous to purify the said reaction raw product, for example by aqueous washing or crystallization. In the case where the compound of Formula 2 is O-acetyl-4-nitro phenol, or O-acetyl-4-nitrosophenol, purification in an aqueous medium enables to hydrolyzate the acetate group to lead to 4-nitro phenol, or 4-nitrosophenol respectively.

The inventors have found that the continuous method according to the present invention leads to an excellent regioselectivity in favor of the para compound, in particular greater than 80%, compared to a batch method.

According to another particular embodiment, the present invention relates to a method as defined above, wherein said method further comprises, after step A of nitration or nitrosation, a step B of hydrogenation of the compound of Formula 2, to obtain:
4-Aminophenol, where R is a benzyl group or a hydrogen atom, or
or O-acetyl-4-aminophenol, where R is an acetate group,

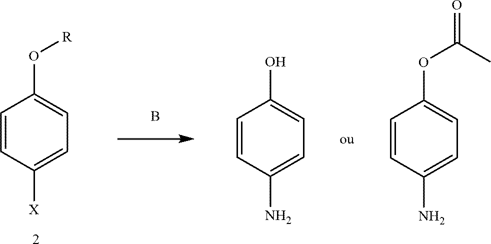

R and X being as defined above,
said step B of hydrogenation being carried out continuously or in batch, preferably continuously, in the presence of hydrogen, a solvent and a catalyst.

The hydrogenation step reduces the nitro group, or the nitroso group to amine. In the case where R represents a benzyl group, this reaction is accompanied by hydrogenolysis of said benzyl group, to obtain p-aminophenol. On the other hand, in the case where R represents an acetate group, O-acetyl-4-aminophenol is obtained, since the acetate group is inert, and is not suppressed under hydrogenation conditions.

Step B of hydrogenation is carried out in the presence of a catalyst that can catalyze a reduction of a nitro or nitroso compound to amine. The catalyst is preferably a heterogeneous catalyst which makes it possible to keep said catalyst within the reactor. For this purpose, the reactor may be equipped with a filtration system, e.g. a sintered filter, at the outlet, to prevent the catalyst from being evacuated with the flows of the reaction crude leaving the reactor. The sintered filter has a porosity of 2 to 50 µm.

According to another particular embodiment, the present invention relates to a method as defined above, wherein step B of hydrogenation is carried out from a compound of Formula 2 wherein X is a nitro group, the compound of Formula 2 being a compound of Formula 2a:

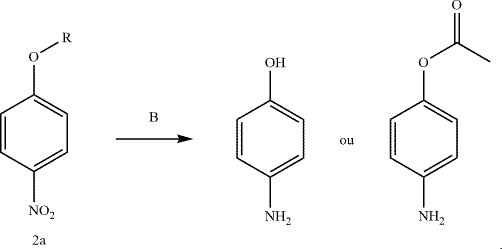

According to another particular embodiment, the present invention relates to a method as defined above, wherein step B of hydrogenation is carried out from a compound of Formula 2 wherein X is a nitroso group, the compound of Formula 2 being a compound of Formula 2b:

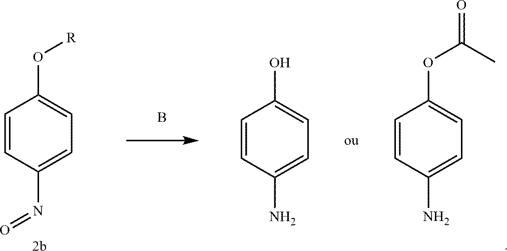

According to another particular embodiment, the present invention relates to a method as defined above, wherein the compound of Formula 2 is p-nitrophenol, R being a hydrogen atom, and X being a nitro group.

According to another particular embodiment, the present invention relates to a method as defined above, wherein the compound of Formula 2 is p-nitrosophenol, R being a hydrogen atom, and X being a nitroso group.

According to another particular embodiment, the present invention relates to a method as defined above, wherein the compound of Formula 2 is mixed with the ortho isomer, in particular wherein the ratio of ortho isomer / compound of Formula 2 is less than 2/8, and is in particular about 1/9.

According to another particular embodiment, the present invention relates to a method as defined above, wherein step B of hydrogenation is carried out in the presence of a catalyst selected from Pd/C, Pt/C and Fe/HCl,

According to another particular embodiment, the present invention relates to a method as defined above, wherein step B of hydrogenation is carried out in the presence of Siliacat Pd(0) as catalyst.

Thus, preferably used a catalyst type Siliacat®, in particular Siliacat Pd (0). Siliacat Pd(0) is a catalyst consisting of Pd trapped in a sol-gel system. Specifically, highly dispersed Pd nanoparticles (uniformly in the range of 4.0 to 6.0 nm), encapsulated in an organosilice matrix. The structure of the catalyst is shown below.

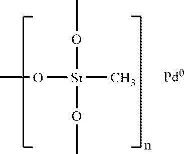

This catalyst is marketed by several companies, including Dichrom GmbH in Germany and Silicycle in Canada.

According to another particular embodiment, the present invention relates to a method as defined above, wherein step B of hydrogenation is carried out in the presence of a solvent selected from ethanol or methanol, in particular ethanol.

According to another particular embodiment, the present invention relates to a method as defined above, wherein step B of hydrogenation is carried out at a temperature of 50 to 130° C., in particular from 80° C. to 100° C.

Below 50° C., the kinetics of the reaction may be too low to be compatible with an industrially viable method. Above 100° C., there is a risk of over-reduction, including reduction of the aromatic ring.

By “50 to 130° C.” is also meant the following ranges: from 60 to 130° C., 70 to 130° C., 80 to 130° C., 90 to 130° C., 110 to 130° C., 60 to 110° C. 80 to 100° C., 70 to 90° C.

According to another particular embodiment, the present invention relates to a method as defined above, wherein step B of hydrogenation is carried out at a hydrogen pressure of 10 to 50 bars, in particular comprised from 15 to 30 bars, in particular about 20 bars.

Below 10 bars, the kinetics of the reaction may be too low to be compatible with an industrially viable method. Above 50 bars, there is a risk of over-reduction, in particular of reduction of the aromatic ring

By “From 10 to 50 bars” is also meant the following ranges: from 15 to 50 bars, from 25 to 50 bars, from 35 to 50 bars, from 10 to 40 bars, from 10 to 30 bars from 15 to 30 bars.

According to another particular embodiment, the present invention relates to a method as defined above, wherein the starting compound of Formula 2 is introduced into the reactor at a concentration of 0.5 to 1.5 M, in particular about 1 M, and at a rate of 5 to 20 ml / minute, in particular 10 or 15 ml / minute.

According to another particular embodiment, the present invention relates to a method as defined above, wherein step B of hydrogenation is carried out in at least two reactors in series, preferably at least three reactors in series and particularly preferably three or five reactors in series.

According to another particular embodiment, the present invention relates to a method as defined above, wherein at least two of the consecutive reactors are of different size.

According to another particular embodiment, the present invention relates to a method as defined above, wherein at least two of the consecutive reactors are of different size, and are of increasing size.

According to this particular embodiment, at least one of the reactors is larger than the size of the previous reactor, at least one of the reactors being the subsequent reactor.

In this configuration, the fluid flow rate is constant and identical between each reactor. The reactor fluid outlet is located at the top of the reactor, as schematized in FIG. 4, and as long as the previous reactor is not filled up to the height of the outlet, the liquid does not leave the reactor. Then the outflow rate is equal to the inflow rate.

This configuration of reactors of increasing size allows a good control of exothermic phenomena, and leads to a very good productivity of the method.

Preferably, the size ratio between the previous reactor and the subsequent reactor is from 1.1 to 3, including 1.5 to 3.

In a particularly preferred way, the present invention relates to a method wherein step B of hydrogenation is carried out in 3 consecutive reactors, of increasing size, in particular with a size ratio of about 1: 1.5: 3.

For example, one can operate with a cascade of reactors of increasing volumes in the following proportions: 1, 1,5, 4.

According to another particular embodiment, the present invention relates to a method as defined above, wherein at least two of the consecutive reactors are of different size, and are of decreasing size.

According to this particular embodiment, at least one of the reactors has a size smaller than the size of the previous reactor. In the subsequent reactor, which has a size smaller than that of the subsequent reactor, the concentration of raw material, i.e. the compound of Formula 2, is lower than in the previous reactor, a part of the said compound of Formula 2 being already converted.

A subsequent smaller reactor makes it possible to increase the load of the catalyst, at a lower cost, in order to compensate for this lower concentration of raw material. In addition, a smaller reactor can be more easily agitated than a larger reactor. This facilitates the dispersion of the catalyst in the reaction medium, which is important when the charge of said catalyst is higher.

Preferably, if the first reactor has a volume R1, the second reactor has a volume R2 between R1 and 0.5 R1 and the third reactor has a volume R3 between 0.8 R1 and 0.4 R1.

For example, one can operate with a cascade of reactors of decreasing volumes in the following proportions: 1:0.75:0.5.

According to another particular embodiment, the present invention relates to a method as defined above, wherein step B of hydrogenation is carried out:
from p-nitrophenol, in the presence of the catalyst SiliaCat Pd(0), or Pt/C
in the presence of SiliaCat Pd(0) or Pt/C as a catalyst,
with ethanol as a solvent,
in three reactors in series,
to obtain p-aminophenol.

Advantageously, the 3 reactors in series are of increasing sizes, as defined above. This embodiment makes it possible to obtain the amine compound with a good yield, in particular with a yield greater than 80%, in particular greater than 95%

According to another particular embodiment, the present invention relates to a method as defined above, wherein step B of hydrogenation is carried out:
from p-nitrosophenol, in the presence of the catalyst SiliaCat Pd(0), or Pt/C
in the presence of SiliaCat Pd(0) or Pt/C as a catalyst,
with ethanol as a solvent,
in three reactors in series,
to obtain p-aminophenol.

Advantageously, the 3 reactors in series are of increasing sizes, as defined above. This embodiment makes it possible to obtain the amine compound with a good yield, in particular with a yield greater than 80%, in particular greater than 95%, in particular greater than 98%

It has been observed, that during step B of hydrogenation, in case of use of a compound of Formula 2 wherein R represents an acetate group, said acetate group migrates to the formed amine function. Thus, the hydrogenation of O-acetyl-4-nitrophenol, or O-acetyl-4-nitrosophenol leads directly to paracetamol.

According to another particular embodiment, the present invention relates to a method as defined above, wherein said method further comprises, after step B hydrogenation, a step C of acylation of p-aminophenol to obtain paracetamol:

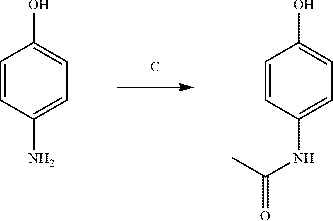

said step C of acylation being carried out continuously or in batch, preferably continuously.

According to another particular embodiment, the present invention relates to a method as defined above, wherein step C of acylation is performed with acetic anhydride as an acylation agent.

According to another particular embodiment, the present invention relates to a method as defined above, which step C of acylation is carried out with acetic acid as an acylation agent, said step C of acylation being carried out under microwaves, in batch, or continuously.

According to another particular embodiment, the present invention relates to a method as defined above, wherein step C of acylation is carried out with an initial ratio of acetic anhydride / p-aminophenol comprised from 1.0 to 1.6, preferably from 1.1 to 1.4.

The use of too much acetic anhydride leads to purification difficulties, compared to the suppression of excess acetic anhydride.

According to another particular embodiment, the present invention relates to a method as defined above, wherein step C of acylation is carried out at a temperature of 60 to 100° C., preferably at about 80° C.

By From “60 to 100° C.” is also meant the following ranges: from 60 to 90° C., from 60 to 80° C., from 60 to 70° C., from 70 to 100° C., from 80 to 100° C., from 90 to 100° C., and from 70 to 80° C.

Step C of acylation is preferably implemented at the same temperature as step B of hydrogenation. For example, if the hydrogenation reaction is implemented at 80° C., the fluid leaving the hydrogenation reactor can be directly injected, hot, into the acylation reactor. Thus, the acylation reaction can be completed in a few minutes, especially in less than 10 minutes, or 5 minutes. Under these conditions it is not necessary to heat the medium further, as the temperature of the fluid is sufficiently high.

According to another particular embodiment, the present invention relates to a method as defined above, comprising:

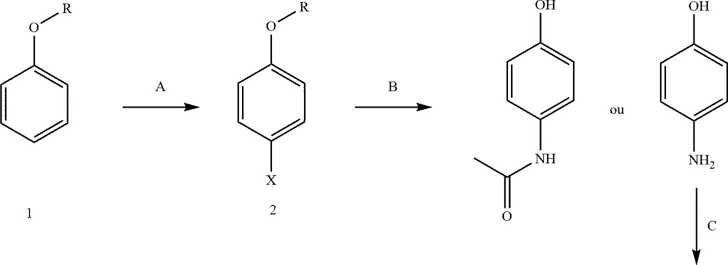

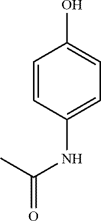

a step A of nitration or nitrosation of a compound of Formula 1, to obtain a compound of Formula 2, said step A of nitration being carried out,
   either continuously, or continuously and under microwaves, or continuously and under ultrasound, or continuously and under microwaves and ultrasound,
   either continuously and optionally under microwaves and / or ultrasound, the nitration agent being sodium nitrite, in the presence of an oxidizing agent, in particular in the presence of HNO₃
   said step A of nitrosation being carried out continuously, the nitrosating agent being sodium nitrite, R and X being as defined above,
a step B of hydrogenation of the compound of Formula 2, to obtain:
   4-Aminophenol, where R is a benzyl group or a hydrogen atom, or
   paracetamol, where R is an acetate group, or said step B of hydrogenation being carried out continuously or in batch, preferably continuously, and
a step C of acylation of 4-aminophenol, to obtain paracetamol, said step C of acylation being carried out continuously or in batch, preferably continuously.

The three steps A, B and C are preferably all performed continuously.

It is understood that step C is to be implemented only if paracetamol is not already obtained at the end of step B of hydrogenation.

According to another particular embodiment, the present invention relates to a method as defined above, comprising:
a step A of phenol nitration, to obtain p-nitrophenol,
   said step A of nitration being carried out continuously and under microwaves,
a step B of hydrogenation of the compound of p-nitrophenol, to obtain p-aminophenol:
   said hydrogenation step B being carried out continuously in three reactors in series, and
a step C of acylation of 4-aminophenol, to obtain paracetamol,
   said step C of acylation being carried out continuously.

This preferred embodiment corresponds to the following diagram:

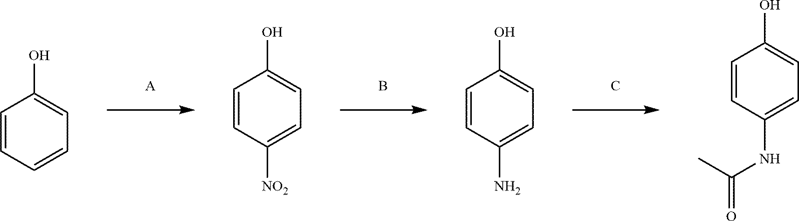

According to another particular embodiment, the present invention relates to a method as defined above, comprising:
a step A of nitrosation of phenol, to obtain p-nitrosophenol,
   said step A of nitrosation being carried out continuously, at a temperature below 10° C.,
a step B of hydrogenation of the compound of p-nitrosophenol, to obtain p-aminophenol:
   said hydrogenation step B being carried out continuously in three reactors in series, and
a step C of acylation of 4-aminophenol, to obtain paracetamol, said step C of acylation being carried out continuously.

This preferred embodiment corresponds to the following diagram:

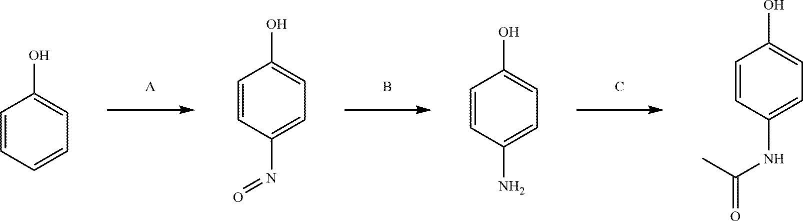

According to another particular embodiment, the present invention relates to a method as defined above, comprising, between at least one step A, B, or C, a purification step, in particular by aqueous washing.

The synthetic intermediates, obtained at the end of steps A of nitration / nitrosation and / or B of hydrogenation, as well as the final product, obtained at the end of step C of acylation can be purified, in order to improve the impurity profile of the method. This may be for example a simple aqueous washing, to remove the residues of acids and salts at the end of step A or C, or a filtration or a bed of coal or zeolite to remove the catalyst residues at the end of step B. Alternatively, more purifying purifications can be implemented such as crystallizations or distillations or continuous liquid/liquid extraction.

According to another particular embodiment, the present invention relates to a method as defined above, further comprising a step D of purification of paracetamol, in particular by continuous distillation, continuous liquid-liquid extraction and or by crystallization, in particular by continuous crystallization.

The final product of the method of the present invention, paracetamol, may be purified by techniques known by those skilled in the art, in order to allow purity compatible with therapeutic use. Purification aims in particular to remove residues of the ortho isomer that may be present in the raw product.

At last, the invention relates to a paracetamol preparation method comprising the successive steps:
1) synthesis of p-nitrophenol from phenol

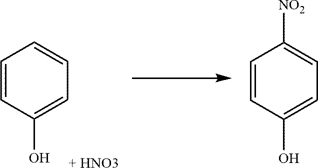

2) synthesis of p-aminophenol from p-nitrophenol

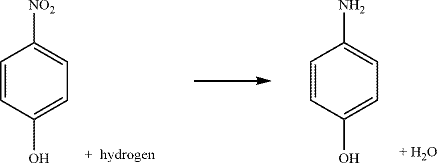

3) synthesis of paracetamol from p-aminophenol

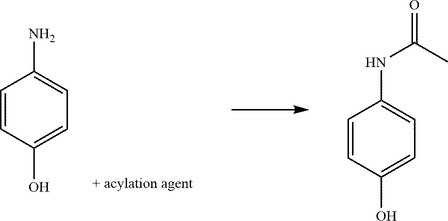

characterized in that steps 1, 2 and 3 are performed continuously and in that step 1) is carried out under microwave.

Indeed, the inventors were able to obtain a regioselectivity of nitration of phenol in para position greater than 60%.

The invention also relates to a paracetamol preparation method comprising the successive steps:
1) synthesis of p-nitrosophenol from phenol

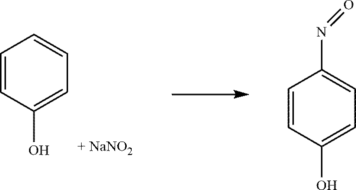

2) synthesis of p-aminophenol from p-nitrophenol

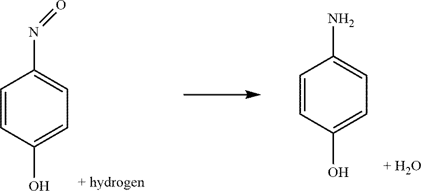

3) synthesis of paracetamol from p-aminophenol

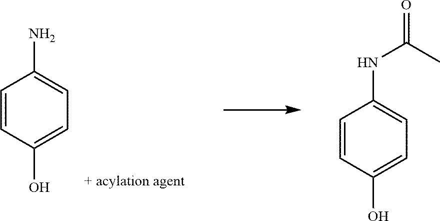

### BRIEF DESCRIPTION OF THE DRAWINGS

FIG. 1 shows different chemical pathways for the production of Paracetamol.

FIG. 2 illustrates the evolution of the temperature of the mixture over time of the reaction mixture as it passes through the circuit formed by several microwave reactors in series, wherein each couple of microwave reactors is interconnected by a cooling circuit.

FIG. 3 schematizes a facility to perform the first step where the phenol/HNOs mixture is introduced into a facility with multiple microwave reactors in series, where each couple of microwave reactors is interconnected by a cooling circuit.

FIG. 4 shows an installation to perform the second hydrogenation stage where several hydrogenation reactors are mounted in series.

FIG. 5 shows a flowchart of the method for the continuous synthesis of paracetamol, including a nitration step.

FIG. 6 shows a flowchart of the method of continuous synthesis of paracetamol, including a nitrosation step.

FIG. 7 shows the conversion of a hydrogenation reaction in a system of 3 reactors in series, according to Example 5.2.

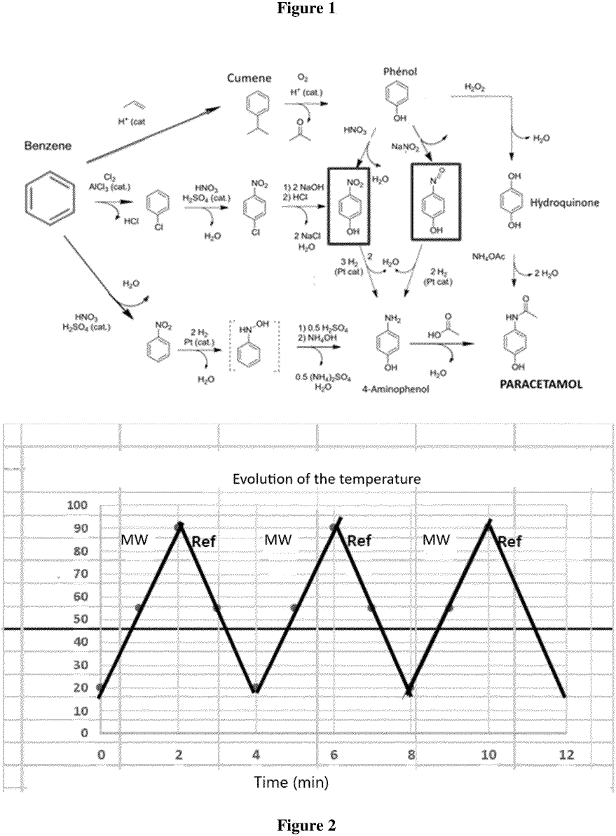

The nitration reaction is carried out by the mixture of phenol and nitric acid.

This reaction is carried out in the presence of a strong acid such as sulfuric acid, hydrofluoric acid, perchloric acid or boron trifluoride. Preferably, this reaction is carried out in the presence of sulfuric acid.

Now, in addition to the microwave step, the inventors have shown that the ratio between the concentration of nitric acid and phenol also has a strong influence on the regioselectivity and obtaining of p-nitrophenol rather than o-nitrophenol. Thus, the use of excess nitric acid promotes the formation of o-nitrophenol. Under such conditions, the inventors were able to obtain up to 82% p-nitrophenol (for 18% o-nitrophenol).

Advantageously the ratio between HNO₃ / Phenol ratio within the starting mixture is between 1.1 and 1.6, preferably between 1.2 and 1.5.

The concentration of the starting mixture in phenol is between 0.2 and 0.6M, preferably between 0.25 and 0.5 M.

The concentration of the starting mixture in HNO₃ is between 0.25 and 0.8M, preferably between 0.3 and 0.7M.

The proportion of water of the starting mixture is between 40 and 95% (in volume relative to the volume of the mixture at this point), preferably between 50 and 90%.

As for step 1, the residence time of the mixture within the microwave reactor is such that the mixture is brought to a temperature between 70 and 110° C., preferably between 80 and 100° C. The change to a higher temperature affects the regioselectivity and tends to increase the proportion of o-nitrophenol.

The inventors were able to show that it is possible to further increase the regioselectivity by increasing the residence time of the mixture in the microwave reactor, but without increasing the temperature.

To do this, the inventors have put in series microwave reactors between which are interspersed cooling circuits.

According to a preferred embodiment, step 1) is carried out in at least two consecutive microwave reactors, preferably at least three consecutive microwave reactors and particularly preferably at least four consecutive microwave reactors, with a cooling circuit between each microwave reactor so as to bring the mixture to a temperature between 20 and 40° C., preferably between 20 and 30° C.

Typically, the residence time in all microwave reactors is between 2 and 20 minutes, preferably between 2 and 15 minutes.

Preferably, each of the microwave reactors (possibly apart from the first) includes a nitric acid feeding system.

Thus, it is possible to maintain the HNO₃ / Phenol ratio within the mixture between 1.1 and 1.6, preferably between 1.2 and 1.5, throughout the nitration reaction (within the different microwave reactors).

FIG. 2 illustrates the evolution of the temperature (°C.) with the time (minutes) of the reaction mixture as it passes through the circuit formed by the microwave reactors (MO) interconnected via a cooling circuit.

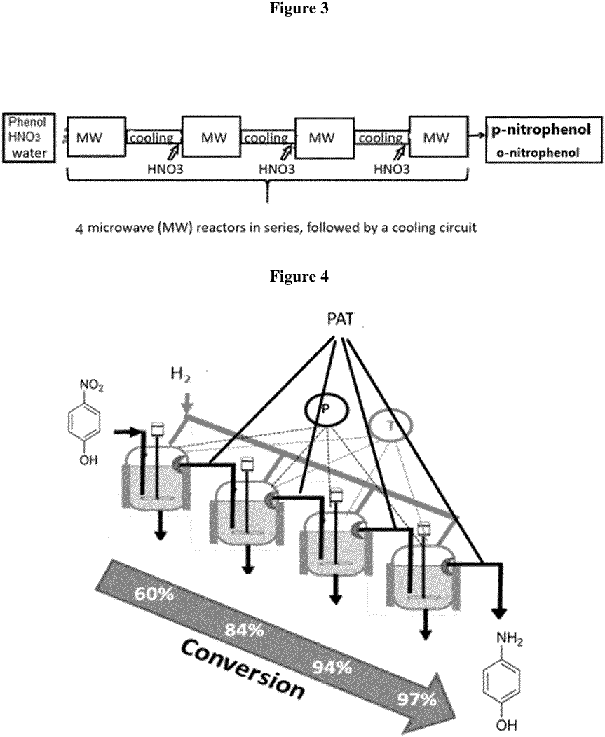

FIG. 3 schematizes an installation to perform the first step where the phenol / HNO₃ mixture is introduced into a first microwave reactor (MO) in which it passes through a first cooling circuit before circulating in a second, third and then fourth microwave reactor to form mostly p-nitrophenol with, each time, a passage through a cooling circuit between each microwave reactor.

To optimize the balance of the reaction, it is necessary to achieve a cooling as fast as possible, typically between 0.5 and 3 minutes, preferably between 1 and 2 minutes.

At the end of step 1), it is possible to simply separate the 2 isomers o-nitrophenol and p-nitrophenol.

Such purification can be carried out by an intermediate step (between steps 1 and 2) of steam distillation of o-nitrophenol (see U.S. Pat. 3,933,929), filtration and washing by an aqueous solution of 70% sulfuric acid and then by water (see patent EP 0626366), solubilization (using the difference in solubility in various solvents of the two isomers, N-pentane to remove o-nitrophenol), ultrafiltration (Yudiarto et al., Separation and Purification Technology, vol. 19, p:103-112, 2000), HPLC (SMB type (Simulated Moving Bed) or VARICOL).

According to a preferred embodiment, p-nitrophenol is purified at the end of step 1) and prior to step 2).

Now it is also possible to start step 2) without purification and separate p-aminophenol from o-aminophenol at the end of step 2).

Concerning the second step of reduction of p-nitrophenol to p-aminophenol, it can be carried out from choice via:

A) addition of dihydrogen under pressure in the presence of catalyst type Pd/C, Pt/C, Fe/HCl or equivalent.

B) addition of a hydrogen donor (e.g. NaBH₄) in the presence of a solid catalyst (gold nanoparticles, etc.).

According to a preferred embodiment, step 2) is carried out by adding dihydrogen under pressure in the presence of catalyst of Pd / C, Pt / C, Fe / HCl or equivalent.

Advantageously, the mixture corresponds to the choice of p-nitrophenol in an aqueous medium in the presence of an acid (preferably sulfuric acid because it gives better yields than hydrochloric acid in particular) or p-nitrophenol in solution in alcohol, preferably ethanol or methanol.

Advantageously, the hydrogenation of p-nitrophenol is carried out in solution of alcohol, preferably in ethanol.

The concentration of the mixture in alcohol is advantageously between 70% and 95% (in volume compared to the volume of the mixture upstream of the hydrogenation reactor), preferably between 80% and 90%.

Preferably, the catalyst used is Pt/C. It is indeed this one that gives the best yields. The catalyst charge within the hydrogenation reactor is included is greater than or equal to 1% (by weight relative to the weight of the mixture within the reactor), preferably greater than or equal to 2% and, particularly preferably, it is equal to 5%.

The pressure within the hydrogenation reactor is advantageously greater than 20 bars.

Preferably, the pressure within the hydrogenation reactor is between 20 and 100 bars, preferably between 20 and 50 bars.

The temperature of the mixture within the hydrogenation reactor is advantageously higher than 80° C.

Preferably, the temperature of the mixture within the hydrogenation reactor is between 80 and 180° C., preferably between 100 and 150° C.

To increase the conversion yield, the inventors put several hydrogenation reactors in series.

According to a preferred embodiment, step 2) is carried out in at least two consecutive hydrogenation reactors, preferably at least three consecutive hydrogenation reactors and particularly preferably at least four consecutive hydrogenation reactors.

Preferably, an on-line analysis of the mixture is carried out between each hydrogenation reactor in order to control the kinetics of the reaction and, therefore, to control the possible deactivation of the catalyst in order to change it when necessary.

FIG. 4 schematizes an installation to perform the second step where the mixture comprising p-nitrophenol in solution in ethanol is introduced into a first hydrogenation reactor comprising solid catalyst (Pt / C) and in which dihydrogen is injected under pressure before passing into a second, third and fourth hydrogenation reactor to form mostly p-aminophenol.

At last, the inventors were able to obtain a conversion yield of p-nitrophenol to p-aminophenol of the order of 97%.

It should be noted that this second step also has a large number of advantages over conventional methods. Indeed, it guarantees high productivity with a small size due to its continuous operation, it offers great safety because of the small volume required for reactors, it allows the use of catalysts to the maximum of their service life.

At the end of step 2), and preferably in the event that p-nitrophenol has not been purified at the end of step 1) and prior to step 2).

According to another preferred embodiment, p-aminophenol is purified at the end of step 2). Such a separation can be achieved simply by the skilled person with regard to his general knowledge, for example by using the differences in solubility between these 2 isomers.

Concerning the third step of acylation of p-aminophenol to paracetamol, it is carried out by the addition to the mixture, and at the exit of the (last) hydrogenation reactor, of an acylation agent.

By acylation agent, both acetic acid and acetic anhydride are considered.

Advantageously, the ratio of acylation agent / p-aminophenol within the mixture, and after the addition of the acylation agent, is between 1 and 10, preferably between 1 and 4.

In the case where the acylation agent is acetic anhydride, the mixture includes alcohol as a solvent, preferably ethanol or methanol.

The acylation reaction is then carried out by heating, preferably heating the mixture to a temperature between 20 and 90° C. and for a time between 0.5 and 10 minutes, and particularly preferably by heating the mixture to a temperature between 20 and 60° C. and for a time between 1 and 4 minutes.

In the case where the acylation agent is acetic acid, it is possible to carry out this acylation in the same way as with acetic anhydride, but without the presence of alcohol and with acetic acid. The temperatures used and the reaction time must then be increased

Typically, the acylation reaction carried out by heating a temperature between 50 and 130° C. and for a time between 1 and 40 minutes, and especially preferably by heating at a temperature between 60 and 100° C. and for a time between 10 and 20 minutes.

Now, the inventors have also demonstrated that this acylation reaction can be carried out very quickly with acetic acid in a microwave reactor. It should also be noted that, in this case, acetic acid can be used as a solvent which considerably simplifies the method since the solvent can be reused by simple distillation of the mixture out of the microwave reactor. Concerning acetic anhydride, in addition to the higher cost, its use then requires the removal of the solvent used (ethanol or methanol)

According to a preferred embodiment, step 3 uses acetic acid and is carried out under microwaves.

Preferably, this step 3 does not use any additional solvent (in addition to acetic acid).

Typically, the p-aminophenol / acetic acid ratio is between ⅕ and ⅒, preferably between ⅙ and ⅑.

To do this, the residence time of the mixture within the microwave reactor is such that the mixture is brought to a temperature between 80 and 120° C., preferably between 90 and 110° C.

Typically, the residence time in all microwave reactors is between 1 and 60 minutes, preferably between 10 and 30 minutes.

At the end of the acylation reaction, paracetamol is continuously purified.

Typically, this purification step can be performed by a simple distillation aimed at removing the solvent.

Advantageously, this purification step may include a washing step, with purified water, especially under inert gas, argon or equivalent.

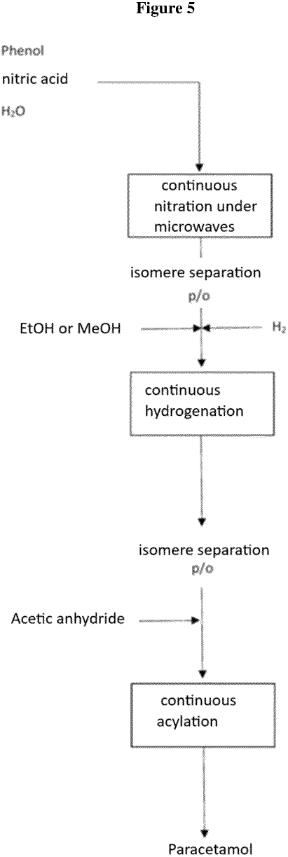

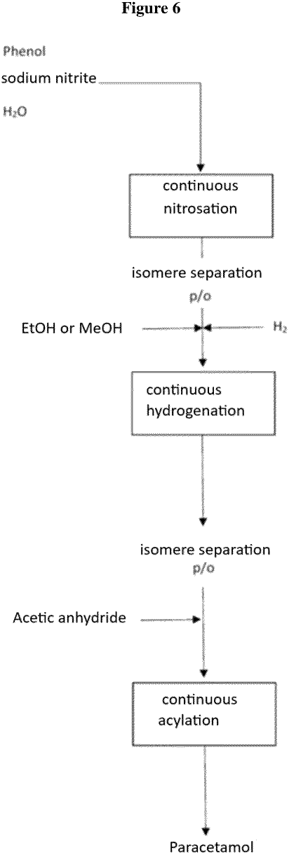

The method according to the invention, of which a flowchart is presented in FIG. 5, makes it possible to synthesize paracetamol with an excellent yield (greater than 70%).

The following examples are provided for illustrative purposes only and cannot limit the scope of the present invention.

### EXAMPLES

### 1) Phenol Nitration:

In a microwave reactor with a volume of 6 ml, 50mg of phenol was introduced with 0.7ml of nitric acid 6% by weight with 1.07ml of H₂O.

The reactor is then set to obtain a temperature of 160° C. for one minute and 30 seconds before cooling at 55° C., before initiating a new heating step at 120° C. for one minute and 30 seconds followed by a new cooling at 55° C.

The results of HPLC analyses have shown that a conversion of phenol to nitrophenol is obtained with a yield of 99.35% overall, but especially with a proportion of nearly 60% p-nitrophenol (and about 40% o-nitrophenol).

Subsequent tests have shown that the faster the cooling step, the more the regioselectivity, and therefore the proportion of p-nitrophenol, increases.

For the continuous nitration reaction, the experiments are carried out in a continuous microwave (SAIREM) with a 2.45GHz wave generator and a 450W power with coaxial transition / waveguide equipped with a cooler.

### 2) Hydrogenation of P-nitrophenol to P-aminophenol

In a continuous hydrogenation reactor (total volume 400ml), separated into different zones each equipped with agitators, the pure solvent is introduced with the catalyst Pt/C. The temperature in the reactor is controlled and maintained at the desired temperature by several thermostatic baths that heat or cool the different areas of the continuous reactor. The hydrogen pressure is kept constant at the desired pressure in each area.

The p-nitrophenol is then continuously introduced into the solvent at a certain rate and concentration.

Output samples are taken to measure conversion and selectivity.

For a flow rate of 800ml/h composed of a solution of p-nitrophenol 0.5M in ethanol, or 13.3ml/min, the catalyst charge Pt/C was 2% (weight/weight of the mixture within the reactor) at a constant temperature of 80° C. in each zone.

The results showed a conversion of 99% with a selectivity of 98% for a residence time of 30 minutes within the reactor.

Optimization is underway regarding the reaction parameters (reaction volume in each zone, catalyst load in each zone, temperature and Pressure H₂ in each zone and overall flow rate).

Already, the results have shown that the Pt/C catalyst provides the best results (at about 1% w/w). Now, for a hydrogenation reactor capable of withstanding a hydrogen pressure of 100 bars and a temperature of 150° C., it is possible to increase the catalyst charge up to 5% (w/w) which induces a sharp increase in productivity by reducing the residence time that can be reduced between 15 to 30 minutes, while maintaining good catalyst activity.

### 3) Acylation of P-aminophenol to Paracetamol:

### 3.1) Temperature Conversion Tests

The tests are carried out in the VAPOURTEC R2+ and R3 type reactor of volume 10ml, which is filled by peristaltic pumps. The samples are then recovered at the exit of the VAPOURTEC to be analyzed by HPLC.

In a first test, the p-aminophenol solution (0.3M in methanol) is injected into the VAPOURTEC at a rate of 5ml/min and at room temperature. Simultaneously, the acetic anhydride solution (0.3M in methanol) is injected into the VAPOURTEC at a rate of 5ml/min at room temperature. The total flow rate is 10ml/min with a passage time of 1mn in the VAPOURTEC.

Analyses showed a p-aminophenol conversion of 99.9%, with a selectivity of 98.7% for paracetamol.

In a second test, the p-aminophenol solution (0.14M in ethanol) is injected into VAPOURTEC at a rate of 5ml/min and at a temperature of 60° C. Simultaneously, the acetic anhydride solution (0.14M in ethanol) is injected into the VAPOURTEC at a rate of 5ml/min at 60° C. The total flow rate is 10ml/min with a passage time of 1min in the VAPOURTEC.

Analyses showed a p-aminophenol conversion of 99.9%, with a selectivity of 98.9% for paracetamol.

In a third test, the p-aminophenol solution (0.14M in ethanol) is injected into VAPOURTEC at a rate of 3.3ml/min and room temperature. Simultaneously, the acetic anhydride solution (0.14M in ethanol) is injected into the VAPOURTEC at a rate of 3.3ml/min at room temperature. The total flow rate is 6.6 ml/min with a passage time of 1.5 minutes in the VAPOURTEC.

Analyses showed a p-aminophenol conversion of 99.9%, with a selectivity of 98.9% for paracetamol.

### 3.2) Microwave Conversion Test

The microwave used was the MONOWAVE 300 (ANTON PAAR) and whose magnetron power is 850 watts. For this one, the power is adapted to the desired temperature.

The various reagents are fed into a 10 mL reactor with stirring that is placed in the microwave enclosure. Once the cycle is complete, the reactor is cooled before taking a sample and performing an HPLC analysis.

In a first test, p-aminophenol is introduced into a solution of acetic anhydride in water (30/70) at a concentration of 7.77 M. The reactor is then introduced into the microwave for 10 seconds and at a temperature of 40° C.

The results showed that a 99.9% conversion of p-aminophenol with a selectivity of 97% for paracetamol was obtained.

In a second test, p-aminophenol is introduced into an acetic acid solution at a concentration of 5M. The reactor is then introduced into the microwave for 20 minutes and at a temperature of 100° C.

The results showed that a 95% conversion of p-aminophenol was achieved with a selectivity of 93.5% for paracetamol.

### 4) Nitration of Phenol by Nitric Acid, Continuously

### 4.1) Uncooled Tubular Continuous Reactor: SAIREM “AVOCADO” Cavity

The reactions were carried out in a 500 mL borosilicate tubular reactor inserted into a cavity of the “AVOCAT” type (SAIREM company) and coupled to a GMS 450 microwave generator that can deliver a maximum power of 450 W thanks to a quartz window transmission. The total volume irradiated is 160 mL.

350 ml of a 0.4 M phenol solution and 0.375 M nitric acid (1.25 eq.) were injected into the cavity at a rate of 16 ml/min. Thus, the time of passage in the irradiated zone was 10 minutes.

The reaction is carried out by microwave irradiation with a power of 250 W, with a microwave generator operating at 2.45 GHz.

Thus, para-nitrophenol was obtained with a productivity of 25 g/h, and a ratio o/p of 20/80.

### 4.2) Cooled Tubular Continuous Reactor: SAIREM “DOWNSTREAM” Cavity

This device consists of a borosilicate tubular reactor (60 mL, internal diameter 12 mm) inserted into a second borosilicate tube (double envelope, internal diameter 23 mm) equipped with coolant inlet and outlet channel. The assembly is inserted into a cavity type “DOWNSTREAM” (company SAIREM) and coupled to a microprobe generator GMS 1000 that can deliver a maximum power of 1000 W thanks to a transmission by quartz window. The total volume irradiated is approximately 10 mL. This device was also equipped with a temperature probe (optical fiber) immersed in the reactor. To cool the internal reactor, a specific oil, of zero dielectric permittivity (therefore transparent to the microwaves), was used. The coolant can be maintained between -10° C. and 0° C. thanks to a cryostat.

An aqueous solution of 0.4 M phenol and 0.375 M nitric acid (1.25 eq.) was injected into the cavity at a rate of 10 ml/min. Thus, the time of passage in irradiated area was 6 minutes.

The test demonstrated that microwave heating with cooling allows perfect control and stable temperature throughout the method.

### 5) Hydrogenation of P-nitrophenol With Siliacat Pd(0) as a Catalyst

### 5.1) Batch Test

The batch reaction was performed on a single closed reactor. The reactor is preloaded with a solution of 6.95 g of p-nitrophenol in 100 mL of EtOH and 0.208 mg of SiliaCat P(0) (reagents purchased from Aldrich and catalyst from SiliCycle). The reactor was then purged by dinitrogen (3 purges, 5-7 bars) and then pressurized with hydrogen (H₂ Alphagaz, Air Liquide) under 15 bars. The agitation is set at 1000 rpm (revolutions per minute rotation per minute).

When the reactor is heated at T = 80° C., a conversion of 86% is obtained in 80 minutes and when the reaction is made at 100° C., a conversion of 88% was obtained in 60 minutes

### 5.2) Continuous Test

The same conditions as those used in Example 2 were used, using the catalyst Siliacat Pd(0) (SiliCycle, Quebec Canada, Ref RD-R815-SiliaCat® Pd0),, at a rate of 0.5 mol%.

A total conversion was achieved in 90 minutes.

### 5.3) Test on 3 Continuous Reactors in Series- Reactors of Increasing Size

The hydrogenation reaction was carried out using 3 reactors in series. The following results were achieved.

| Parameters | Operating conditions R1 | | Operating conditions R2 | | Operating conditions R3 (modélisation) | |
|---|---|---|---|---|---|---|
| Volume | 0,1 | L | 0,15 | L | 0,4 | L |
| Flow rate | 12 | ml/mn | 12 | ml/mn | 12 | ml/mn |
| Pressure | 20 | Bar | 12 | Bar | 5 | Bar |
| Temperature | 100 | °C. | 110 | °C. | 130 | °C. |
| Mcata Siliacat Pd | 0,70% | | 1,70% | | 2% | |
| Conc p-nitrophenol input | 1 | mol/l | 0,64 | mol/l | 0,163 | mol/l |
| Conc p-nitrophenol output | 0,64 | mol/l | 0,16 | mol/l | 0,011 | mol/l |
| Conversion | 0,36 | | 0,73 | | 0,93 | |
| Overall conversion % | 35,95 | | 83,62 | | 98,85 | |

In the table above, the amount of catalyst “Mcata” is expressed in mol%.

A productivity of 3.7kg/L/day of p-aminophenol was achieved with 3 reactors in series.

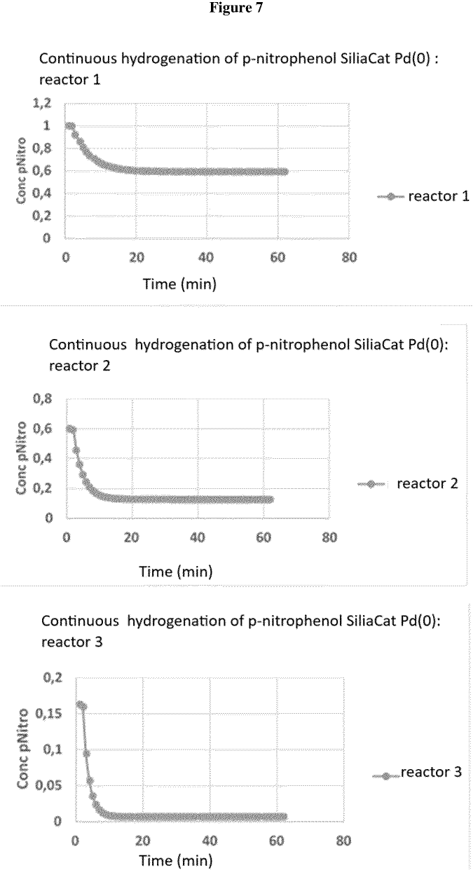

FIG. 7 shows the conversions for each reactor.

### 5) Hydrogenation of P-nitrophenol on a Cascade of Two or Three Perfectly agitated continuous reactors

### 5.1) Batch Focus

The reaction in batch mode is carried out on a single closed reactor. The reactor is pre-charged with a solution of 6.95 g of p-nitrophenol in 100 mL of EtOH and 9.75 mg of Pt/C (Sigma Aldrich). The reactor is then purged of dinitrogen (3 purges, 5-7 bars) and then pressurized with hydrogen (H₂ Alphagaz, Air Liquide) under 15 bars. The agitation is fixed at 1000 rpm and the reactor is heated to 80° C. by its double envelope for 1h20. At the end of the reaction, the reactor is inerted by a purge of the dinitrogen and the reaction medium is analyzed by HPLC (reverse phase, column C18). The analysis shows a conversion of 92% of p-nitrophenol to p-aminophenol without trace of reaction co-product.

### 5.2) Cascade Reaction

The same device used in Example 5.1, is reused to perform the reaction on a cascade of two perfectly agitated continuous reactors. The outlet line of the first reactor, always equipped with a 5 µm filtering candle to maintain the catalytic loading of the autoclave constant, is connected at the entrance of a second reactor at any point similar to the first. Both reactors are charged with 20 mg Pt/c 10% w/w (Sigma Aldrich). A conversion of 50% is simulated in the first reactor (2.72 g of p-aminophenol for 3.48 g of p-nitrophenol) and a conversion of 75% is simulated in the second reactor (4 g of p-aminophenol per 1.8 g of p-nitrophenol). Under the conditions described above, but with a slightly decreasing pressure (80° C., 15 bars, 1000 rpm in the first reactor; 80° C., 12 bars, 1000 rpm in the second reactor), the cascade is fed by a solution of p-nitrophenol in ethanol (0.3 M) at a flow rate of 3 mL/min (passage time, 30 minutes per reactor) for 5 hours. The withdrawal valve of the second reactor is set to have an output flow rate approximately equal to the input flow rate. No events occur during the 5 hours of reaction. Samples are taken every 4 minutes. HPLC analyses show that the conversion oscillates between 70 and 83% for 20 minutes before stabilizing around 80% without co-product formation.

In another case, a third reactor is connected to the cascade. Similarly, this reactor is loaded with 20 mg Pt/C, and a starting conversion of 90% is simulated (4.9 g p-aminophenol per 695 mg p-nitrophenol). Under the conditions described above (80° C., 1000 rpm, 15 bars; 12 bars; 10 bars), the waterfall is fed for 4 hours at a flow rate of 4 mL/min (passage time 25 minutes). No events occur. At the reactor outlet, samples are taken every 4 minutes. HPLC analyses show that the conversion oscillates between 80 and 96% for 20 minutes before stabilizing at 95% for 4 hours.

### 6) Phenol Nitrosation and Hydrogenation - Batch Protocol

### 6.1) Nitrosation

To a solution of HCl 35% (40 ml) under air, stirring at T = 0° C., was added drop by drop a solution of NaNO₂ (42% in water, 2 eq.). The solution turned orange and released a small amount of orange gas. A solution of phenol in water (80% in water, 1 g, 1 eq.) was added drop by drop to the solution (Final phenol concentration = 0.3M). The solution gradually turned black and the mixture had become denser. After 30 minutes, an HPLC analysis shows that phenol has been totally consumed. The mixture was diluted in 500 mL of H2O and was extracted by AcOEt (3*250 mL). The organic phases were collected, dried on Na₂SO₄, filtered and evaporated dry to obtain a mixture of 2-nitrosophenol, and 4-nitrosophenol.

### 6.2) Hydrogenation of Pure P-nitrosophenol a Mixture of Para and Ortho-nitrosophenol

The dry crude mixture, obtained in Example 6.1, was solubilized in MeOH, Pt / (C) (mass %) was suspended, the mixture was then under hydrogen (1 atm) under stirring. After 2 hours, the mixture showed no trace of 2-nitrosophenol and 4-nitrosophenol. The solution was filtered on celite, and evaporated dry to obtain a mixture of 2-aminophenol and 4-aminophenol in a ratio of o / p = 10/90.

Two further hydrogenation tests of pure p-nitrosophenol were performed under pressure (P=15 bars) at T=80° C., using the catalyst Pt/C and the catalyst SiliaCat Pd(0). It was obtained a conversion of the order of 99.9% with an excellent yield of 99.8% (control by HPLC)

### 7) Phenol Nitrosation - Continuous Protocol

The same ratios used in Example 6.1 were tested continuously. However, after 5 minutes of residence time in the continuous reactor, the aqueous solution of phenol was added, and after a residence time of 5 minutes nitrosophenol was obtained continuously. The extraction was done in batch to carry out batch hydrogenation, according to the conditions of Example 6.2, to obtain a mixture of 2-aminophenol and 4-aminophenol in a ratio of o / p = 10 / 90.

1-47. (canceled)

48. A method for preparing paracetamol, wherein the method comprises a step A of nitration, or nitrosation, of a compound of Formula 1 with a nitration agent, or a nitrosating agent suitable for obtaining a compound of Formula 2: 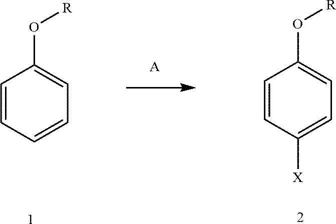
wherein R represents:
a hydrogen atom,
a protective group selected from a benzyl or acetate, and wherein X represents a nitro group or a nitroso group, said step A of nitration being carried out continuously:
either under microwaves,
either under ultrasound,
either under microwave followed by ultrasound,
or, optionally under microwaves and / or ultrasound, the nitration agent being sodium nitrite, in the presence of an oxidizing agent, in particular in the presence of HNO₃, said step A of nitrosation being carried out continuously:
the nitrosating agent being sodium nitrite.

49. The method according to claim 48, wherein step A is a nitration step, R being as defined above, and X being a nitro group, to obtain a nitro compound wherein the compound of Formula 2 has the structure of a compound of Formula 2a: 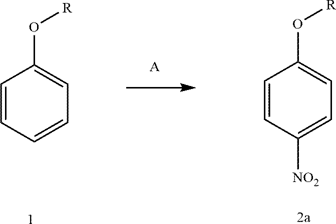
.

50. The method according to claim 48, wherein step A is a nitrosation step, R being as defined above, and X being a nitroso group, to obtain a nitroso compound wherein the compound of Formula 2 has the structure of a compound of Formula 2b: 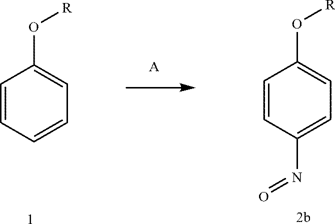
.

51. The method according to claim 48, wherein step A of nitration is carried out with a nitration agent selected from HNO₃ and NaNO2, to obtain a compound of Formula 2 wherein X is a nitro group.

52. The method according to claim 48, wherein step A of nitration comprises:
a feeding system to a reactor with a solution, in particular aqueous of the compound of Formula 1, in particular in concentration of about 0.4 M, and with the nitration agent, in solution, in particular aqueous, in particular at a concentration of about 0.3 to 0.4 M, to obtain a reaction medium,
the formation of the compound of Formula 2.

53. The method according to claim 48, wherein step A of nitration is carried out with an initial ratio of nitration agent / compound of Formula 1 comprised from 1.1 to 1.6, preferably from 1.2 to 1.5.

54. The method according to claim 48, wherein step A of nitration is carried out in at least two microwave reactors in series, preferably at least three microwave reactors in series and particularly preferably at least four microwave reactors in series
comprising, between each reactor in series, a cooling step so as to adjust the temperature to a temperature of 20 to 40° C., preferably from 20 to 30° C.

55. The method according to claim 48, wherein step A of nitrosation is carried out in an acidic medium, in particular in an aqueous solution of hydrochloric acid or sulfuric acid.

56. The method according to claim 48, comprising a step A of nitrosation, wherein a reactor is powered by an aqueous solution of the compound of Formula 1, and by NaNO₂ in aqueous solution in an acid, in particular in hydrochloric acid.

57. The method according to claim 48, wherein step A of nitrosation is carried out at a temperature below 10° C., in particular comprising from -5 to 5° C., in particular at a temperature of about 0° C.

58. The method according to claim 48, wherein step A of nitration, or nitrosation leads to the formation of the compound of Formula 2, in particular p-nitrophenol or p-nitrosophenol, in particular p-nitrosophenol, with a regioselectivity greater than 60%, in particular greater than 80%, in particular wherein the ratio of ortho / compound of Formula 2 is less than 2/8, and is in particular about ⅑.

59. The method according to claim 48, wherein said method further comprises, after step A of nitration or nitrosation, a step B of hydrogenation of the compound of Formula 2, to obtain:
4-Aminophenol, where R is a benzyl group or a hydrogen atom, or
or O-acetyl-4-aminophenol, in the case where R is an acetate group 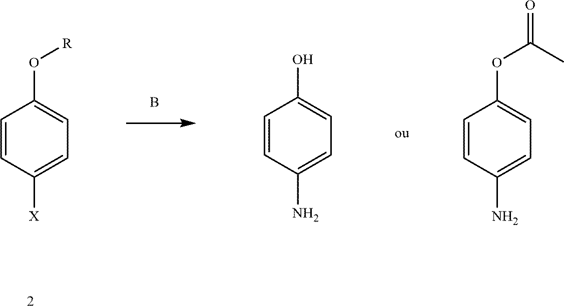
R and X being as defined above,
said step B of hydrogenation being carried out continuously or in batch, preferably continuously, in the presence of hydrogen, a solvent and a catalyst.

60. The method according to claim 59, wherein step B of hydrogenation is carried out in the presence of a catalyst selected from Pd / C, Pt / C and Fe / HCl, optionally
wherein step B of hydrogenation is carried out in the presence of Siliacat Pd (0) as a catalyst,
wherein step B of hydrogenation is carried out in the presence of a solvent selected from ethanol or methanol, in particular ethanol,
wherein step B of hydrogenation is carried out at a temperature of 50 to 130° C., in particular from 80° C. to 100° C.,
wherein step B of hydrogenation is carried out at a hydrogen pressure of 10 to 50 bars, in particular comprising from 15 to 30 bars, in particular about 20 bars.

61. The method according to claim 59, wherein step B of hydrogenation is carried out in at least two reactors in series, preferably at least three reactors in series and particularly preferably three or five reactors in series.

62. The method according to claim 59, wherein said method further comprises, after step B of hydrogenation, a step C of acylation of p-aminophenol to obtain paracetamol: 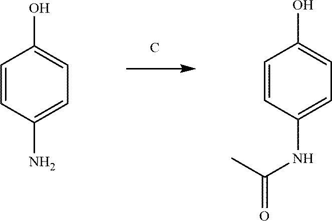
said step C of acylation being carried out continuously or in batch, preferably continuously.

63. The method according to claim 62, wherein step C of acylation is carried out with acetic anhydride as an acylation agent, optionally wherein step C of acylation is carried out at a temperature of 60 to 100° C.

64. A method for preparing paracetamol comprising: 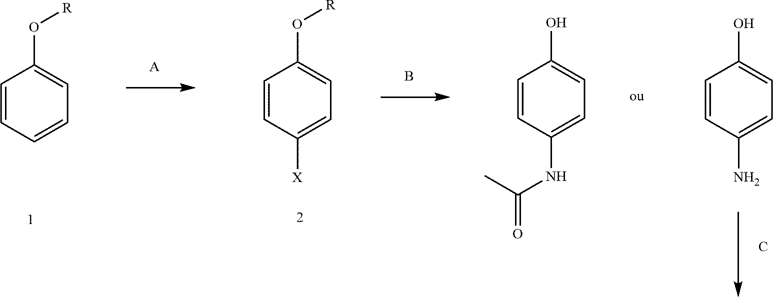 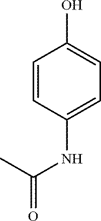
a step A of nitration or nitrosation of a compound of Formula 1, to obtain a compound of Formula 2,
said step A of nitration being carried out,
either continuously, or continuously and under microwaves, or continuously and under ultrasound, or continuously and under microwaves and ultrasound,
either continuously and optionally under microwaves and / or ultrasound, the nitration agent being sodium nitrite, in the presence of an oxidizing agent, in particular in the presence of HNO₃
said step A of nitrosation being carried out continuously, the nitrosating agent being sodium nitrite, R and X being as defined above,
a step B of hydrogenation of the compound of Formula 2, to obtain:
4-Aminophenol, where R is a benzyl group or a hydrogen atom, or paracetamol, where R is an acetate group, or
said step B of hydrogenation being carried out continuously or in batch, preferably continuously, and
a step C of acylation of 4-aminophenol, to obtain paracetamol,
said step C of acylation being carried out continuously or in batch, preferably continuously.

65. The method according to claim 64, comprising:
a step A of phenol nitration, to obtain p-nitrophenol,
said step A of nitration being carried out continuously and under microwaves,
a step B of hydrogenation of the compound of p-nitrophenol, to obtain p-aminophenol:
said step B of hydrogenation being carried out continuously in three reactors in series, and
a step C of acylation of 4-aminophenol, to obtain paracetamol,
said step C of acylation being carried out continuously.

66. The method according to claim 64, comprising:
a step A of nitrosation of phenol, to obtain p-nitrosophenol,
said step A of nitrosation being carried out continuously, at a temperature below 10° C.,
a step B of hydrogenation of the compound of p-nitrosophenol, to obtain p-aminophenol:
said step B of hydrogenation being carried out continuously in three reactors in series, and
a step C of acylation of 4-aminophenol, to obtain paracetamol,
said step C of acylation being carried out continuously.

67. The method according to claim 64, further comprising a step D of purification of paracetamol, in particular by continuous distillation, continuous liquid-liquid extraction and or by crystallization, in particular by continuous crystallization.
